(19) United States
(12) Patent Application Publication
(10) Pub. No.: US 2013/0298273 A1
(43) Pub. Date: Nov. 7, 2013

(54) Watermelon Double Grafting Methods

(71) Applicants: Yen Ming Chang, Lodi, CA (US); Francisco Javier López Fernández, Almeria (ES)

(72) Inventors: Yen Ming Chang, Lodi, CA (US); Francisco Javier López Fernández, Almeria (ES)

(73) Assignee: NUNHEMS B.V., Nunhem (NL)

(21) Appl. No.: 13/838,318

(22) Filed: Mar. 15, 2013

Related U.S. Application Data
(60) Provisional application No. 61/644,086, filed on May 8, 2012.

(30) Foreign Application Priority Data
May 7, 2012 (EP) 12166925.3

(51) Int. Cl. A01H 5/08; A01G 1/06

(52) U.S. Cl. CPC A01H 5/08; A01G 1/06
USPC 800/260; 800/308; 47/6

(57) ABSTRACT

The invention relates to the field of watermelon grafting. In particular, double grafted watermelon seedlings and plants are provided and methods for producing such double grafted seedlings and plants.

### CROSS-REFERENCE TO RELATED APPLICATIONS

This application claims priority to European Application No. EP 12166925.3, filed May 7, 2012, and U.S. Provisional Patent Application to 61/644,086, filed May 8, 2012, the contents of each are hereby incorporated by reference in their entirety.

### FIELD OF THE INVENTION

The present invention relates to the field of watermelon production. Provided are methods for producing grafts of a diploid watermelon pollinizer plant (2n=2x=22) and a triploid watermelon plant (2n=3x=33) onto one single rootstock. Further, different methods of producing triploid watermelon fruits (and optionally diploid fruits) in the field, comprising the use of such “double grafted” watermelon plants, is provided herein. Further “double grafted” watermelon seedlings, comprising three genetically different plant parts (a rootstock, a triploid scion and a diploid scion) and plants grown from such seedlings, are provided.

In a further aspect, methods for producing grafts of two diploid watermelon scions, e.g. two pollinizer scions, onto a single rootstock are provided. In a further aspect methods for producing grafts of two triploid watermelon scions onto a single rootstock are provided. Thus also “double grafted” watermelon seedlings, comprising a rootstock and two triploid scions, or a rootstock and two diploid scions, and plants grown from such seedlings, are provided.

### BACKGROUND OF THE INVENTION

Seedless watermelon (Citrullus lanatus (Thunb.) Matsum. And Nak.) production involves using pollen from diploid male parent plants to fertilize flowers of tetraploid (2n=4x=44) maternal parent plants. Pollination of the tetraploid flowers with diploid pollen leads to hybrid F1 seeds which are triploid (Kihara, 1951, Proceedings of American Society for Horticultural Science 58: 217-230; Eigsti 1971, Hort Science 6: 1-2). The triploid hybrid plants, grown from these F1 seeds, are self-infertile as they produce sterile pollen due to chromosome imbalance (Fehr, 1987). The triploid hybrids, therefore, need to be pollinated by a diploid pollenizer to produce watermelon fruit. Triploid plants are, therefore, interplanted with pollenizer plants for fruit production. The “seedless” fruit produced after pollination on the triploid hybrid plant are often not truly seedless, but may contain some undeveloped, small, pale seeds, which are edible.

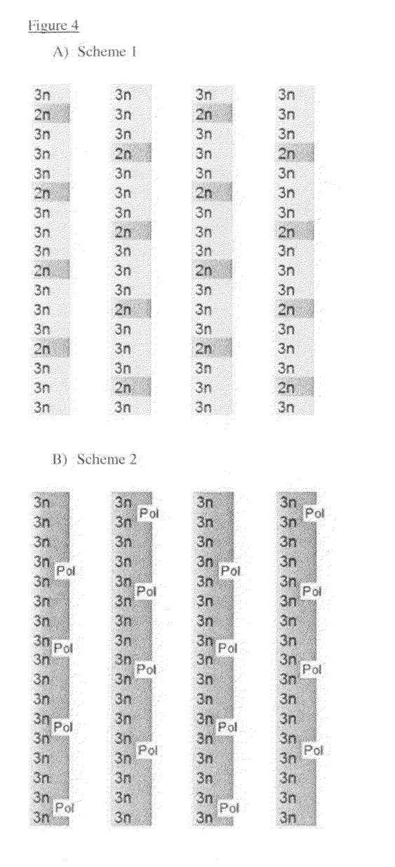

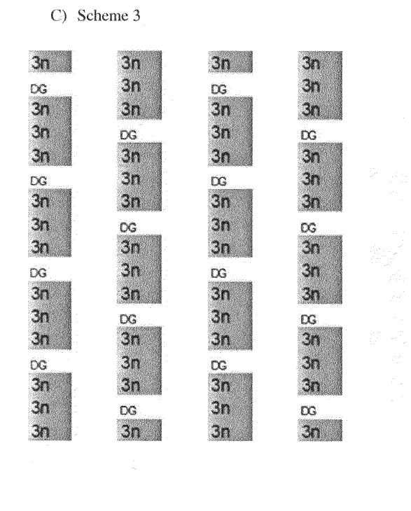

For optimal seedless watermelon fruit set, sufficient viable pollen is required. Plants are generally planted at a ratio of 1 pollenizer per every 2, 3, 4 or even 5 triploid plants. Triploid plants and pollenizers are up to date either planted in separate rows (e.g. 1 row of pollenizer and 2-4 rows of triploids), or interplanted within rows (e.g. planting 1 pollenizer plant in between 2, 3, 4 or 5 triploid plants in the same row), or interplanted in narrow pollinizer rows between rows of triploids (see US 2006/0168701 Table 2 and FIG. 4).

Grafting of triploid scions or diploid pollenizers scions to a rootstock of a different plant is a common method used in watermelon production. In Spain for example all triploids and all diploid pollenizers are grafted. Grafted seedling plants are often prepared by dedicated nurseries, who prepare the grafted plants either by hand or automated methods, depending on the grafting method used. Trays with seedling transplants are then provided to the grower and planted in the field for triploid fruit production.

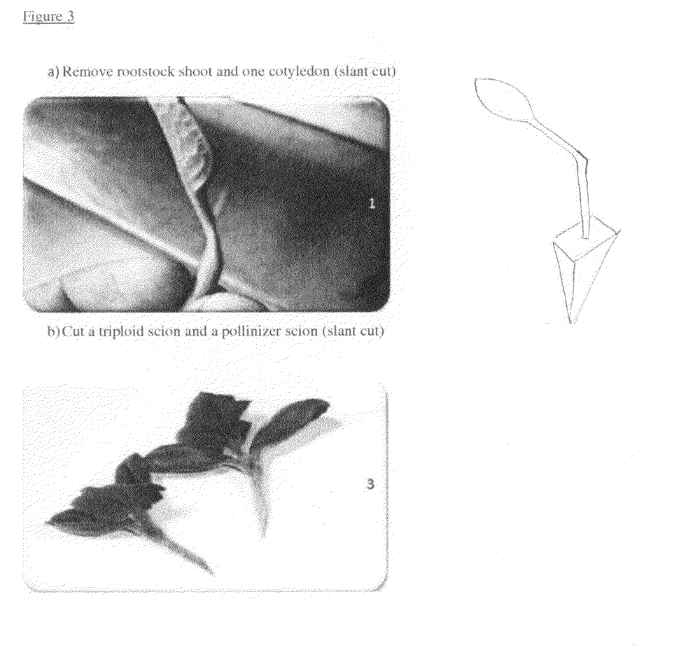

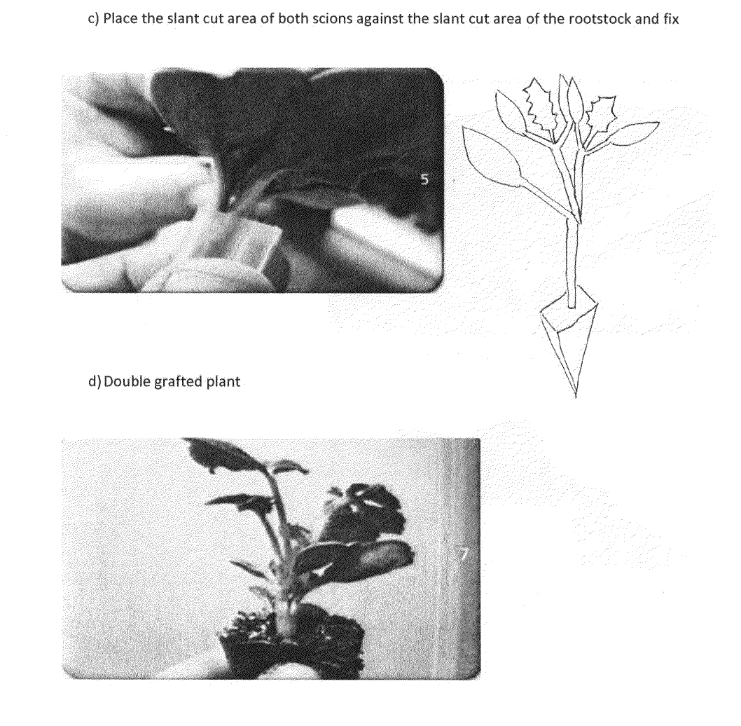

Generally, triploid seedlings and diploid pollenizer seedlings are provided in different trays and are only arranged in specific patterns when planted into the field. However, the seedlings may be arranged in the trays in the same order as they are arranged in the field. For example US2011/0203501 describes in FIGS. 3 and 4 how mechanical seeders place a pre-arranged pattern of triploid seeds alone and both a triploid seed and a diploid seed in the wells of a tray. For example, a seedling tray may comprise three wells of each one triploid seedling per well followed by one well of one triploid seedling and one diploid pollenizer seedling, and so forth. As seeds are sown in the wells, these seedlings are not grafted.

Grafting watermelons has a number of advantageous, as is explained in Davis et al. (2008), Critical Reviews in Plant Sciences Vol. 27, “Cucurbit Grafting”, page 50-74, incorporated herein by reference. The main advantage of grafting is that rootstocks can be used which provide or enhance resistance against soilborn diseases, especially when genetic or chemical approaches for disease management are not available or not sufficient. Thus, disease susceptible watermelon scions can be grafted onto disease resistant rootstocks for watermelon production. Apart from providing resistance against fungi and viruses, the use of grafting can also increase tolerance against different abiotic stresses such as cold/low temperature tolerance, drought tolerance, salinity tolerance, flooding/water tolerance and can have beneficial effects on e.g. growth, yield, nutrient uptake, plant vigor, fruit size and fruit quality.

A number of different grafting methods have been described, each having their own advantages and disadvantages. The most common methods are described in Davis et al. (2008), supra, and are amongst others the following:
1) Tongue Approach/Approach Graft,
2) Hole insertion/Terminal/Top Insertion Graft,
3) One Cotyledon/Slant/Splice/Tube Graft and
4) Cleft/Side Insertion Graft.

Also a ‘double graft’ method has been described, but in this method a watermelon scion is grafted onto a rootstock, which in return is grafted onto another rootstock.

No methods have been described where two scions, especially a triploid watermelon scion and a diploid pollinizer scion, or two diploid watermelon scions or two triploid watermelon scions, are grafted onto one single rootstock, referred herein as “double graft” or “double grafting”.

### SUMMARY OF EMBODIMENTS OF THE INVENTION

It is an object of the invention to provide methods for double grafting and to provide double grafted seedlings and double grafted watermelon plants grown from such seedlings. It is a further object to provide methods of using such double grafted seedlings in triploid, seedless watermelon production.

### DETAILED DESCRIPTION OF EMBODIMENTS OF THE INVENTION

### Definitions

The verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, unless it is clear from the context that only one is referred to.

As used herein, the term “plant” includes the whole plant or any parts or derivatives thereof, preferably having the same genetic makeup as the plant from which it is obtained, such as plant organs (e.g. harvested or non-harvested fruits, leaves, etc.), plant cells, plant protoplasts, plant cell-or tissue-cultures from which whole plants can be regenerated, plant calli, plant cell clumps, plant transplants, seeds from which the plant can be grown and seeds produced by the plant, seedlings, plant cells that are intact in plants, plant clones or micropropagations, or parts of plants, such as plant cuttings, embryos, pollen, ovules, fruits (e.g. harvested tissues or organs), flowers, leaves, clonally propagated plants, roots, stems, root tips, grafts (scions and/or root stocks) and the like. Also any developmental stage is included, such as seedlings, cuttings prior or after rooting, etc.

“Grafting” refers to the method of joining of (genetically) different plant parts, especially scions and rootstocks, together so that they grow as a single plant. A grafted seedling or a grafted plant is a seedling or plant (produced by grafting) consisting of such different plant parts and which grows as one plant.

A “non-grafted” watermelon seedling or plant refers to a seedling or plant grown from a seed (without grafting).

A “single grafted” watermelon seedling or “single grafted” watermelon plant refers to a grafted seedling or plant consisting of a single watermelon scion (e.g. a triploid watermelon scion or a diploid watermelon scion) joined with a genetically different rootstock such as a gourd or squash rootstock, another watermelon rootstock, a transgenic rootstock, etc.

A “double grafted” watermelon seedling or a “double grafted” watermelon plant is herein a grafted seedling or plant comprising two watermelon scions grafted onto a single rootstock. In one aspect two genetically different watermelon scions, namely a triploid watermelon scion and a diploid watermelon scion, are grafted onto a genetically different rootstock, such as a gourd or squash rootstock, another watermelon rootstock, a transgenic rootstock, etc. In another aspect two triploid watermelon scions, or two diploid watermelon scions, are grafted onto a genetically different rootstock, such as a gourd or squash rootstock, another watermelon rootstock, a transgenic rootstock, etc.

A “scion” or “watermelon scion” refers to the part of a watermelon seedling that is grafted onto the rootstock and that develops into the aerial part of the plant.

“Rootstock” or “watermelon compatible rootstock” refers to the root system and stem onto which the watermelon scions are grafted and which provides the root system for the grafted seedling and grafted plant. It is noted that during the grafting process, the rootstock root system may be removed, which later grows back to develop a functional root system of the grafted seedling. Thus, when referring to the rootstock during the grafting method, this rootstock may be with or without the root system. When referring to the rootstock of the grafted seedlings or plants, the re-grown root system is encompassed.

“Rootstock shoot” refers to the true leaves developing from the shoot apical meristem tissue on the rootstock seedling. “Rootstock meristem” refers herein to the shoot apical meristem tissue or growing tip of the rootstock seedling, from which aerial portions of the plant, such as true leaves, develop.

A “transplant” or “seedling transplant” refers to a watermelon seedling which is at a developmental stage and condition so that can be transplanted into the field or greenhouse for growth , fruit production and harvest. The word transplant or seedling transplant can thus encompass single-grafted, double grafted or non-grafted seedlings.

“Healing” or “healing process” refers to the time and conditions required for the scion(s) and rootstock to establish a vascular connection. Depending on the grafting method used, different post-grafting conditions may be desired to achieve good healing, such as high relative humidity (RH) (at least about 85% RH, at least about 90% or 95% RH or even 100% RH), temperature above 20° C., such as a temperature of about, or in between about, 24 ° C. to 28 ° C. (e.g. about 24° C., about 25° C., about 26° C., about 27° C. or about 28° C.) and low light intensity (e.g. shade or darkness) for one or two or more days (e.g. 1, 2, 3, 4, 5, 6, 7 or more days). Healing is generally achieved in a healing chamber.

As used herein, the term “variety” or “cultivar” means a plant grouping within a single botanical taxon of the lowest known rank, which can be defined by the expression of the characteristics resulting from a given genotype or combination of genotypes.

“Diploid plant” refers to a plant, vegetative plant part(s), or seed from which a diploid plant can be grown, having two sets of chromosome.

“Triploid plant” refers to a plant, vegetative plant part(s), or seed from which a triploid plant can be grown, having three sets of chromosomes.

“Tetraploid plant” refers to a plant, vegetative plant part(s), or seed from which a tetraploid plant can be grown, having four sets of chromosomes.

“Pollenizer plant” or “pollenizer” refers to the (inbred or hybrid) diploid plant or seedling, or parts thereof (e.g. a scion), suitable as pollenizer for inducing fruit set on triploid plants. A pollenizer plant is, thus, able to lead to good fruit set (and good triploid fruit yield) of triploid plants, by producing an appropriate amount of pollen at the appropriate day-time and for an appropriate period of time, e.g. at least during peak flowering time of the triploid female plants. A good triploid fruit yield is, for example, a yield comparable to the yield obtainable when using Polimax (produced by Nunhems) as pollenizer.

“Dual purpose pollenizer” refers to a pollenizer plant which also produces edible diploid fruits on the pollenizer plant itself (through self-pollination) and also is suitable to be used as a pollenizer in triploid (seedless) watermelon production. This definition is independent of whether or not the plant is actually being used as a pollenizer in triploid fruit production, i.e. it can also be used for diploid fruit production on its own.

The term “edible” is used herein to refer to fruits “marketable” for human consumption, especially fresh consumption of the fruit flesh. The fruits have at harvest at least good, preferably very good flavor properties (i.e. taste and odor). To have good flavor properties the fruits preferably have an average level of Total Soluble Solids of at least about 7.5% or more, especially at least about 8%, 9%, 9.5%, 10%, or more. Good fruit flesh color is also an important criterion for marketability for human consumption. For red-fleshed fruits it is an embodiment that the flesh color has an average RHS rating of at least 39 or above. If red-fleshed fruits are measured on a scale of 1 (white) to 10 (dark red), the fruits have an average rating of at least 6, 7 or more.

“Hybrid triploid plant” is a triploid plant grown from hybrid, triploid seed obtained from cross fertilizing a male diploid parent with a female tetraploid parent.

“Seedless fruit” are triploid fruit which contain no or few mature seeds. The fruit may contain one or more small, edible, white ovules. Plants which produce seedless fruit may herein be referred to as “seedless”.

“Interplanting” refers to the combination of two or more types of seeds and/or transplants sown or transplanted on the same field, especially the transplanting of “double grafted” watermelon seedlings in the same field as non-grafted or single grafted triploid hybrid seedlings (for seedless fruit production on the triploid plants and/or triploid scions). For example, the double grafted watermelon seedling may either be planted in separate rows or interplanted with the triploid plants in the same row (e.g. in hills within each row). Double grafted seedlings may also be planted in between rows of triploids. Also seeds of (non-grafted) triploid hybrids may be sown in the field and double grafted seedlings may be transplanted in separate rows or the same rows (into spaces left during seeding) or between rows of triploids. As mentioned, the triploid hybrids may be non-grafted or single-grafted (i.e. the transplants may comprise a rootstock of a different plant).

“Planting” or “planted” refers to seeding (direct sowing) or transplanting seedlings (plantlets) into a field by machine or hand.

“Vegetative propagation” refers to propagation of plants from vegetative tissue, e.g. by in vitro propagation or grafting methods (using scions).

Throughout this document “average” and “mean” are used interchangeably and refer to the arithmetic mean.

In one embodiment the invention provides a “double grafted” watermelon seedling, comprising two watermelon scions and one (watermelon-compatible) rootstock. Thus, a plant seedling is provided comprising two scions of the species Citrullus lanatus joined to a single rootstock by grafting. The two watermelon scions are preferably selected from one triploid watermelon scion and one diploid watermelon scion. Thus, a plant seedling is provided wherein one of the scions is from a triploid watermelon plant and the other scion is from a diploid watermelon plant.

However, the two watermelon scions may alternatively be selected from two diploid scions or two triploid scions. All aspects and methods herein which refer to a diploid and a triploid watermelon scion are understood to alternatively also refer to two diploid or to two triploid watermelon scions, instead of the triploid and diploid scion, and these are equally embodiments of the invention. Two diploid watermelon scions may for example be two watermelon pollenizer scions, whereby pollen production can be increased and/or extended over a longer period of time in the field. The two pollenizer scions may be from the same or from different pollenizer lines or varieties, i.e. they may be genetically identical or genetically different. For example, one watermelon scion may be a diploid pollenizer scion and the other scion may be a dual purpose pollenizer scion. When using two triploid watermelon scions joined to one rootstock, these may also be from the same, or from two different, triploid watermelon lines or varieties. The use of two triploid watermelon scions may be used to increase overall triploid fruit yield in the field.

The triploid watermelon scion(s) is (are) in one embodiment from a triploid hybrid watermelon variety, such as but not limited to commercial triploid hybrid varieties, e.g. Nunhems seedless varieties, such as Boston F1, Selecta F1, Constitution F1, Estel Deluxe F1, Revolution F1, Freedom F1, Style F1, Ivona F1, Pixie F1, Bobbie F1, Valdoria F1, Vanessa F1; Syngenta's seedless varieties, such as Fascination, Melody, Summer King, Sweet Delight, TRI-X Brand 212, TRI-X Brand 313, TRI-X Brand Palomar, Imagination, Amarillo, Matrix; Seminis seedless watermelon varieties, such as Apollo, Cooperstown, Cronos, Majestic, Olympia, Wrigley, or others.

In one embodiment the triploid hybrid scion used to produce the double grafted seedling is the same triploid hybrid line or variety with which the double grafted seedlings are interplanted for triploid fruit production.

The diploid watermelon scion(s) is (are) in one embodiment from a watermelon pollenizer. Diploid pollenizers are for example pollenizers Polimax F1 or Jenny F1 (Nunhems), Red Star F1 (Nunhems), Super-pollenizers SP-1, SP-2, SP-3, SP-4 or SP-5 (Syngenta), Companion (Seminis), Escort-4 (Gold Seed Co. US 2009/0288183) or others. The pollenizer may produce marketable fruits (seeded) and may be an open pollinated or hybrid diploid. Alternatively, the pollenizer may produce non-marketable fruits. In one embodiment the fruits produced on the diploid pollenizer part of the plant are preferably distinguishable from the fruits produced on the triploid hybrid part of the plant, so that they can either be not harvested or sorted out and optionally marketed separately, as they are seeded. Thus, for example the rind pattern, or fruit size at maturity is preferably different. The diploid watermelon scion(s) may also be from a dual purpose pollenizer.

The scion of the diploid pollenizer is preferably from a pollenizer which is a suitable pollenizer providing sufficient pollen during the right stage to pollinate the female flowers of the triploid hybrid with which the double grafted seedlings are to be interplanted for triploid fruit production. Thus, in one embodiment the diploid pollenizer scion preferably produces (when further developed after transplanting) a large number of male flowers at the appropriate time during flowering of the triploid (non-grafted or single-grafted) triploid hybrid with which the double grafted seedlings are to be interplanted for triploid fruit production. Optionally the pollenizer may also be a suitable pollenizer for fertilizing the female flowers produced on the double grafted plants, i.e. on the triploid scion and/or on the diploid scion. The double grafted plants may thus produce seedless triploid fruits and seeded, diploid fruits, either of which, or both of which, may be marketable. Which fruits the double grafted plants produce obviously depends on the two scions. If the scion is a diploid and a triploid scion, then both seeded (diploid) and seedless (triploid) fruits will be produced. If the scions are two diploid scions, only seeded fruit will be produced and if the scions are two triploid scions only seedless (triploid) fruits will be produced on the double grafted plants.

To produce the triploid and/or diploid scions for grafting, the seeds of the triploid watermelon and/or the seeds of the diploid pollenizer are germinated under suitable conditions and allowed to grow until both cotyledons have fully developed and preferably at least one true leaf starts to develop or until at least one true leaf is present (fully developed) and optionally the second true leaf starts to develop. The preferred developmental stage of the scions depends on the grafting method used.

The timing of sowing of the triploid watermelon seeds, diploid pollenizer seeds and rootstock seeds is such that the seedlings reach a suitable developmental stage by the time the grafting method is to be carried out. This is further illustrated elsewhere herein. Usually the rootstock seed will be sown several days after the triploid and the pollenizer seeds.

When the watermelon seedling has the desired size, the hypocotyl is cut below the cotyledons, i.e. between the cotyledons and the root ball (leaving however sufficient hypocotyl length) and the roots are discarded. Depending on the grafting method used, the cut may be in wedge form (pointed, with cut-areas on both sides of the hypocotyl) or at an angle (e.g. an angle of 35° to)45° on only one side of the hypocotyl (at a slant). In one embodiment the cut on the hypocotyl, exposing the vascular tissue, is preferably about the same or similar size as the wounded area of the rootstock where the hypocotyl and rootstock are joined, as explained further below.

As mentioned, the scions are from watermelon, i.e. from the species Citrullus lanatus.

The rootstock onto which the two scions are grafted may be any watermelon-compatible rootstock. The rootstock is preferably genetically different than both scions. Preferably the rootstock used confers enhanced resistance or improved tolerance onto the double grafted seedling and/or plants against one or more soil born diseases, such as fungal diseases (e.g. Fusarium wilt, Verticillium dahlia, Phomopsis sclerotiodes, Phytophthora), viral diseases and/or nematode damage. The rootstock may confer other benefits onto the double grafted seedlings and/or plants, such as increased cold- or heat tolerance, increased salinity tolerance, increased flooding/water stress tolerance, increased fruit yield, extended harvest period, increased vigour, improved performance in exhausted or marginal soils, etc.

In one embodiment the rootstock used to create the double grafted seedlings is the same rootstock as is used in the single-grafted triploids in the field. E.g. if a specific Langenaria rootstock is used in creating single-grafted triploid hybrids, the same Langenaria rootstock is used for creating the double grafted seedlings. However, in certain embodiments the triploids that are interplanted with the double grafted plants are non-grafted.

The rootstock has preferably good compatibility with the triploid and/or diploid scions.

In one embodiment the rootstock is selected from the group consisting of: a Cucurbita species rootstock, a bottle gourd rootstock (Lagenaria siceraria, synonym L. vulgaris), wax gourd rootstock, an interspecific hybrid of two Cucurbita species, an interspecific squash hybrid, a Citrullus species rootstock (e.g. Citrullus lanatus), a transgenic rootstock (e.g. comprising a transgene that confers disease resistance or another beneficial trait). Watermelon compatible rootstocks are also listed in Table 5 of Davis et al. (2008, supra).

The Cucurbita species rootstock may be selected from the species Cucurbita maxima, Cucurbita moschata (squash), Cucurbita pepo (pumpkin), an interspecific hybrid between C. maxima and C. moschata (also referred to as Interspecific hybrid squash).

The rootstock may for example be a rootstock selected from Nunhems rootstock varieties Macis F1 (species Lagenaria siceraria), Nun 3001 RT F1 (species Lagenaria siceraria), Shintosa Camelforce F1 (interspecific Cucurbita hybrid), Ercole F1 (interspecific Cucurbita hybrid); or from Syngenta's rootstock varieties Emphasis (Langenaria type rootstock) and Strong Tosa (Interspecific squash hybrid); or from other watermelon compatible rootstocks.

In one embodiment the double grafted plant comprises an interspecific Cucurbita hybrid rootstock (e.g. Shintosa Camelforce F1), a triploid watermelon scion of a commercial triploid variety, such as Pixie F1, and a diploid watermelon scion, such as a commercial diploid pollenizer (e.g. Jenny F1, or Pollimax) or a dual purpose pollenizer or commercial diploid watermelon variety, such as Red Star F1.

A number of different grafting methods are provided herein, which can be used to join the two scions with the rootstock, as will be described below.

Generally, the cut hypocotyl surface of each of the two scions is brought into contact with a wounded or cut surface of the rootstock, to result in the joining of three (in one embodiment genetically distinct) plant parts. Depending on the method used, the three plant parts may or may not need to be held together by a clamp, tube, clip or other mechanical fastening means. The joined section(s) is (are) then allowed to heal, which may require specific post-grafting conditions (e.g. high relative humidity for a few days) and double grafted seedlings are then acclimated (or acclimatized) to natural conditions before being transplanted into the field or greenhouse.

In one embodiment (transplanting) trays are provided comprising the double grafted seedlings, allowing transport and transplanting into the field. In one embodiment each well of the tray comprises a double grafted seedling according to the invention.

In another embodiment, only every second, third, fourth or fifth well comprises a double grafted seedling according to the invention. The wells in-between the double grafted seedlings each comprise a triploid hybrid seedling, which may be either non-grafted or single-grafted. A tray comprising a 2:1 ratio of triploid hybrids (X) to double grafted seedlings (D) may thus look like this:

| | | | | | | |
|---|---|---|---|---|---|---|
| D | X | X | D | X | X | D |
| X | D | X | X | D | X | X |
| X | X | D | X | X | D | X |
| D | X | X | D | X | X | D |
| X | D | X | X | D | X | X |
| X | X | D | X | X | D | X |
| D | X | X | D | X | X | D |
| X | D | X | X | D | X | X |
| X | X | D | X | X | D | X |
| D | X | X | D | X | X | D |
| X | D | X | X | D | X | X |

Alternatively a tray comprising a 2:1 ratio of triploid hybrids (X) to double grafted seedlings (D) may comprise the triploid hybrids and the double grafted seedlings in different rows, such as:

| | | | | | |
|---|---|---|---|---|---|
| D | X | X | D | X | X |
| D | X | X | D | X | X |
| D | X | X | D | X | X |
| D | X | X | D | X | X |
| D | X | X | D | X | X |
| D | X | X | D | X | X |
| D | X | X | D | X | X |
| D | X | X | D | X | X |
| D | X | X | D | X | X |
| D | X | X | D | X | X |
| D | X | X | D | X | X |

The arrangement of seedlings in the tray is thereby in one embodiment the same as the arrangement in the field and transplanting can take place in a single pass. This reduces the occurrence of errors during transplanting and saves labour time.

Thus, a tray is provided comprising triploid hybrid watermelon seedlings and the double grafted seedling according to the invention in a ratio of 5:1, 4:1, 3:1, 2:1 or 1:1.

In one embodiment each of 5, 4, 3 or 2 consecutive wells contain a triploid hybrid watermelon seedling followed by one well containing a double grafted seedling according to the invention. In another embodiment each triploid hybrid is followed by a double grafted seedling. In a further embodiment all wells contain double grafted seedlings.

In another embodiment the tray comprises rows of only triploid hybrids and rows of only double grafted seedlings, whereby the ratio of triploid rows to double grafted seedling rows is 5:1, 4:1, 3:1 or 2:1, or optionally 1:1. Thus every 6^{th}, 5^{th}, 4^{th}, 3^{rd}, or 2^{nd} row may consist of double grafted seedlings only.

The transplanting trays may be any type of tray known in the art. For example, commonly trays may comprise 24, 32, 54, 72, 98, 128, 200 or 242 wells per tray. They may be composed of materials such as Styrofoam, polystyrene, plastic (hard or flexible), etc. The wells may contain soil or a mix of e.g. 50 to 65% high grade peat and 35 to 50% horticultural vermiculite or horticultural perlite, or other growth compositions/growth mixes.

Seedlings are ready for transplanting when the roots are sufficiently developed to permit removal from the well with the entire soil or growing mix volume intact. This will generally require about four to six weeks from sowing or seeding, depending on well size, light and temperature conditions.

In a further embodiment not only the double grafted seedling plants, but also mature plants grown from the double grafted plants according to the invention and plant parts, such as fruits or fruit parts obtainable from the double grafted plants are provided.

As mentioned above, different grafting methods are provided herein, by which double grafted seedlings and plants according to the invention can be made. These are non-limiting examples, as the skilled person can develop further or alternative methods based on the teaching herein. Also it is understood that the exact details of the protocols (such as the number of days before scions are cut) may vary slightly, depending on seedling species or variety or line, seedling age, seedling vigour, skill and experience of the person carrying out the method, growth conditions (light, temperature, RH, etc.), etc.

In any of the grafting methods described herein the rootstock itself does not require a root system and the grafting method can be carried out just with the upper part of the rootstock seedling. The roots may be removed either by hand (e.g. with a cut e.g. about 1, 2, or 3 cm below the rootstock cotyledons) or by machine. The roots of the rootstock will re-grow from the rootstock stem after the grafted seedling is planted into a growth composition. Thus, when reference is made to the rootstock in the grafting methods, this may be either a rootstock with roots or without roots. The rootstock re-grows during healing (and thereafter) and the double grafted plants have a fully functional rootstock.

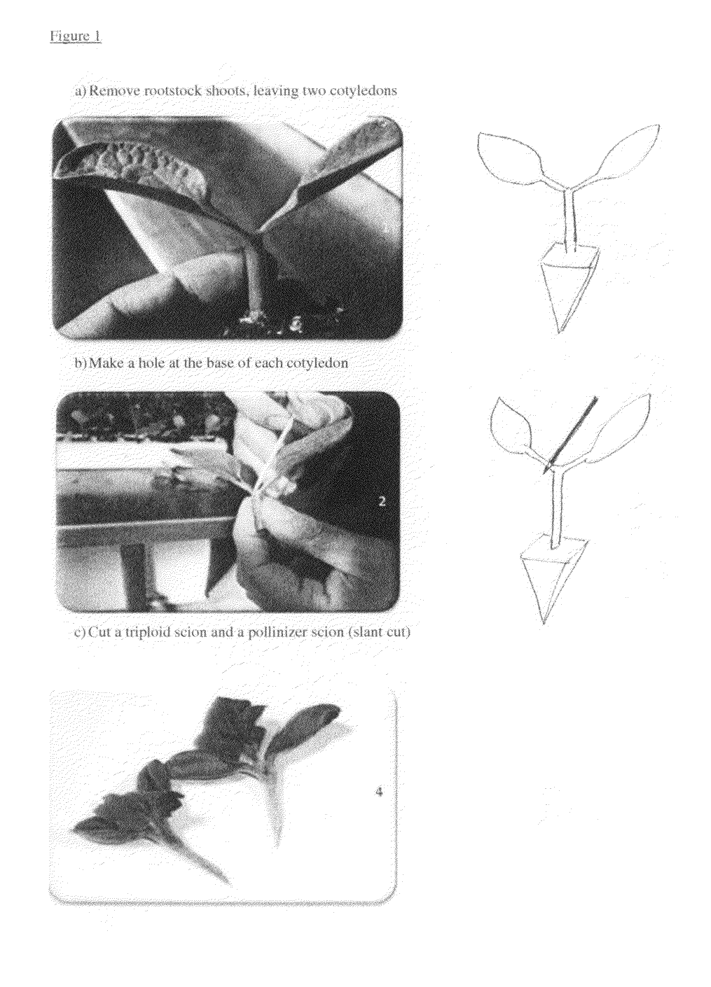

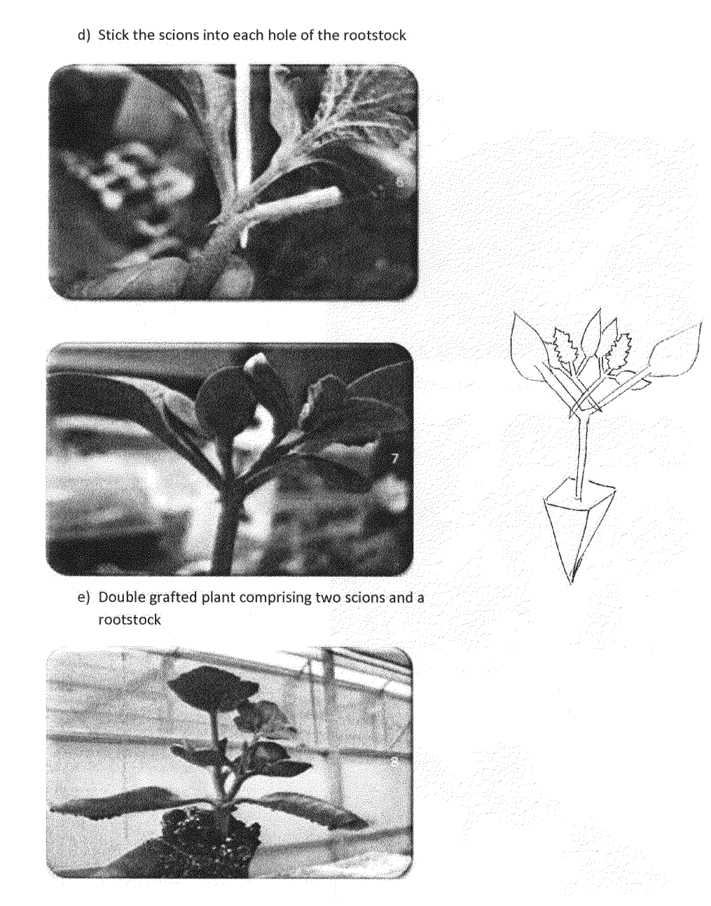

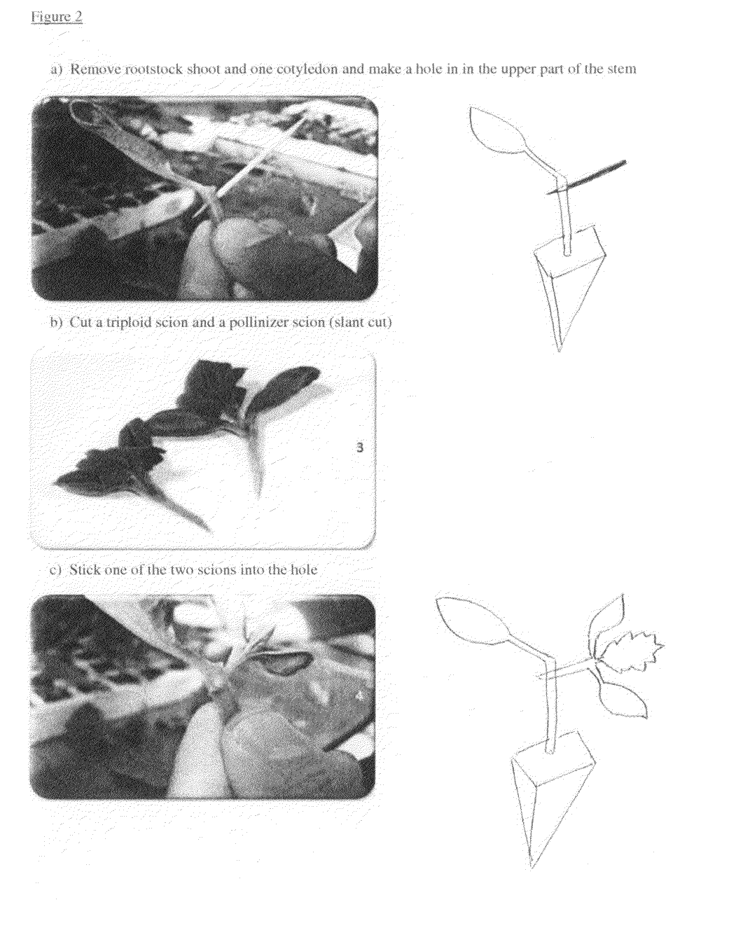

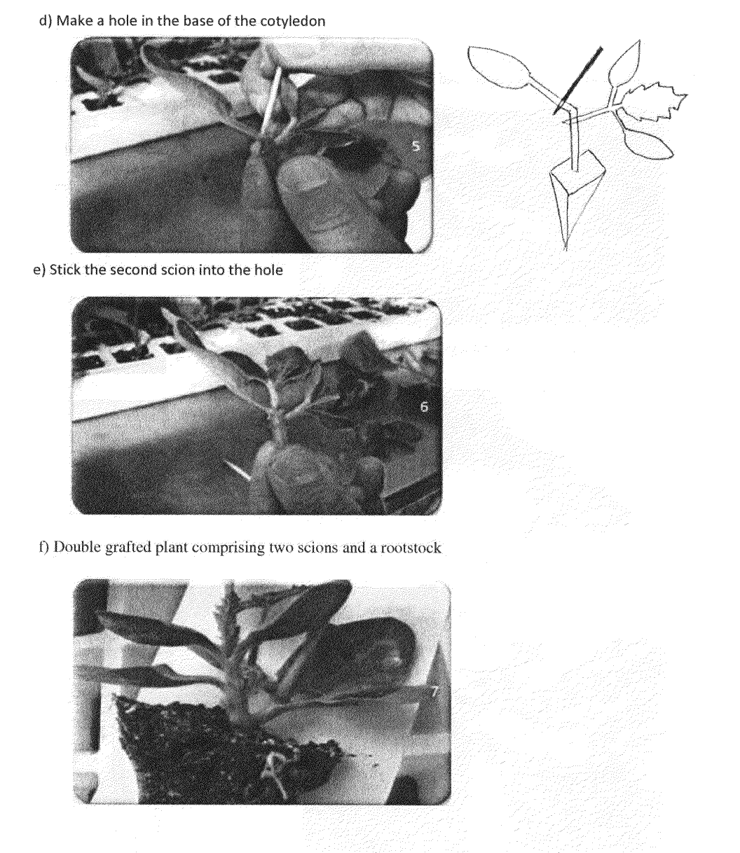

Four methods are encompassed herein:
1) Double insertion graft method, see FIG. 1;
2) Double insertion/stem graft method, see FIG. 2;
3) One cotyledon double graft method, see FIG. 3;
4) Double stem insertion graft method.

### Double Insertion Graft Method (FIG. 1)

Provided is a method for making a double grafted watermelon seedling, comprising the steps of:
a) Providing a rootstock comprising two cotyledons,
b) removing the rootstock meristem and shoot,
c) making a hole at the base of each cotyledon,
d) inserting a watermelon scion into one hole and inserting a watermelon scion into the other hole of the cotyledons, and
e) allowing healing to occur.

In one aspect, step d) comprises inserting a triploid watermelon scion into one hole (at the base of one cotyledon) and inserting a diploid watermelon scion (e.g. a diploid pollenizer scion) into the other hole (at the base of the other cotyledon). In another aspect, both scions are diploid watermelon scions (e.g. pollenizer scions) or both scions are triploid watermelon scions. Thus, in one aspect step d) comprises inserting a diploid watermelon scion (e.g. a diploid pollenizer scion) into one hole (at the base of one cotyledon) and inserting a diploid watermelon scion (e.g. a diploid pollenizer scion) into the other hole (at the base of the other cotyledon). And in another aspect step d) comprises inserting a triploid watermelon scion into one hole (at the base of one cotyledon) and inserting a triploid watermelon scion into the other hole (at the base of the other cotyledon).

Step a) involves sowing a suitable rootstock seed and allowing the seedling to grow until both cotyledons have fully developed, and preferably the first true leaf has developed, and optionally the second true leaf starts to develop, or until at least one true leaf is present. The rootstock meristem and shoots are then removed and a hole is made in step b) through each rootstock cotyledon base. This can suitably be done with a toothpick or other pin.

To make the watermelon scions for grafting, a triploid watermelon seed is sown and a diploid watermelon seed is sown (or two triploid, or two diploid, watermelons are sown) and these are allowed to grow until they have two cotyledons and preferably the first true leaf is developing or has developed, and optionally the second true leaf is starting to develop. The hypocotyl is then cut below the cotyledons with a slant (using for example a razor blade) and each scion is stuck through one of the holes made at the base of the cotyledons of the rootstock. The cut side of the scions is preferably oriented towards the stem of the rootstock to ensure proper connection of vascular tissue between scions and rootstock.

The distance where the hypocotyl is cut below the cotyledons is for example about 0.5, 1.0, 1.5, 2.0, 2.5, 3.0 or 3.5 cm.

Optionally the hypocotyl of the scions may also be cut with two or more slant cuts, to create a wedge or pointed tip on the scion.

No fastening means is required to keep the scions in place. The double grafted seedlings are then placed into a healing chamber for about 1, 2, 3, 4, 5, 6, 7 or more days to allow healing to occur, before acclimatization and transplanting can be carried out. In one embodiment the seedlings are placed in the healing chamber for at least about 3 days.

In one aspect the method further comprises growing the double grafted seedlings into mature plants, i.e. acclimatizing the seedlings and transplanting the seedlings into the field.

### Double Insertion/Stem Graft Method (FIG. 2)

Provided is a method for making a double grafted watermelon seedling, comprising the steps of:
(a) Providing a rootstock comprising two cotyledons,
(b) removing the rootstock meristem and shoot and optionally one cotyledon,
(c) making a hole at the base of (one of) the cotyledon(s) and making a hole in the upper part of the rootstock stem,
(d) inserting a watermelon scion into one hole and inserting a watermelon scion into the other hole, and
(e) allowing healing to occur.

In one aspect, step d) comprises inserting a triploid watermelon scion into one hole and inserting a diploid pollenizer watermelon scion into the other hole. In another aspect, both scions are diploid scions (e.g. pollenizer scions) or both scions are triploid scions, as described for the method above (FIG. 1).

Step a) involves sowing a suitable rootstock seed and allowing the seedling to grow until both cotyledons have fully developed and preferably the first true leaf has developed and optionally the second true leaf starts to develop, or until at least one true leaf is present. The rootstock meristem region and rootstock shoot is removed. Optionally one of the cotyledons may also be removed (step b). The removal can be carried out with a razor blade and by doing one or two (or several) cuts. Preferably, the cut removing the meristem and shoot is not at a slant but near horizontal, so that the sides of the upper part of the stem are undamaged. The rootstock shoot may also be removed by hand, instead of a cut, if it is long enough. The final rootstock thus contains roots (optional), a stem (without shoot and/or meristem tissue) and one or two cotyledons. A hole is made in step c) through the upper part of the stem and through the base of one cotyledon. This can suitably be done with a toothpick or other pin. Alternatively, instead of a hole, a small cut (or slit) may be made through the stem, for example with the point of a razor blade.

To make the scions for grafting, a triploid watermelon seed is sown and a diploid watermelon seed is sown (or two triploid, or two diploid, watermelons are sown) and these are allowed to grow until they have two cotyledons and preferably the first true leaf is developing or has developed, and optionally the second true leaf is starting to develop. The hypocotyl is then cut below the cotyledons with a slant (using for example a razor blade) and one scion is stuck through the hole made in the upper part of the rootstock stem and the other scion is placed through the hole made at the base of the cotyledon of the rootstock. In one embodiment the diploid scion (e.g. the diploid pollenizer) is placed through the hole in the stem and the triploid watermelon scion is placed through the hole at the base of the cotyledon.

The distance where the hypocotyl is cut below the cotyledons is for example about 0.5, 1.0, 1.5, 2.0, 2.5, 3.0 or 3.5 cm.

Optionally the hypocotyl of the scions may also be cut with two or more slant cuts, to create a wedge or pointed tip on the scion.

No fastening means is required to keep the scions in place. The double grafted seedlings are then placed into a healing chamber for about 1, 2, 3, 4, 5, 6, 7 or more days to allow healing to occur, before acclimatization and transplanting can be carried out. In one embodiment the seedlings are placed in the healing chamber for at least about 3 days.

In one aspect the method further comprises growing the double grafted seedlings into mature plants, i.e. acclimatizing the seedlings and transplanting the seedlings into the field.

### One Cotyledon Double Graft Method (FIG. 3)

Provided is a method for making a double grafted watermelon seedling, comprising the steps of:
a) Providing a rootstock comprising two cotyledons,
b) removing one cotyledon and the rootstock meristem (or shoot) with a slant cut,
c) providing a triploid watermelon scion and a diploid watermelon scion having a slant cut hypocotyl end,
d) placing the slant cut hypocotyl end of the triploid watermelon scion and of the diploid pollenizer watermelon scion onto the slant cut surface of the rootstock,
e) attaching fastening means around the two scions and the rootstock, and
f) allowing healing to occur.

As mentioned previously, instead of a triploid and diploid scion, also two triploid or two diploid scions may be used in this method, thereby providing a method for making a double grafted watermelon seedling, comprising the steps of:
a) Providing a rootstock comprising two cotyledons,
b) removing one cotyledon and the rootstock meristem (or shoot) with a slant cut,
c) providing two watermelon scions having a slant cut hypocotyl end,
d) placing the slant cut hypocotyl end of the two scions onto the slant cut surface of the rootstock,
e) attaching fastening means around the two scions and the rootstock, and
f) allowing healing to occur.

In this method the scions may be selected from the group consisting of i) one triploid watermelon scion and one diploid watermelon scion, ii) two diploid watermelon scions and iii) two triploid watermelon scions.

Step a) involves sowing a suitable rootstock seed and allowing the seedling to grow until both cotyledons have fully developed and the first true leaf has developed and optionally the second true leaf starts to develop, or until at least one true leaf is present. The rootstock meristem region and rootstock shoot is removed, as is one of the cotyledons (step b). The removal can be carried out with a razor blade and by doing one or two (or several) cuts. Preferably only one cut is used to remove the cotyledon and rootstock meristem and shoot. Preferably, the cut removing the cotyledon and shoot/meristem is at a slant, e.g. at an angle of about 40°, 41°, 42°, 43°, 44°, 45°, 46°, 47°, 48° (degrees). Thus, if one takes the horizontal cut through the hypocotyl to be at a 90° degree angle, the slant cut is at the aforementioned angles.

To make the scions for grafting, a triploid watermelon seed is sown and a diploid watermelon seed is sown (or two diploid seeds, or two triploid seeds, are sown) and these are allowed to grow until they have two cotyledons and preferably the first true leaf is developing or has developed, and optionally the second true leaf is starting to develop. The hypocotyl is then cut below the cotyledons with a slant (using for example a razor blade). Preferably the angle of the slant cut is about the same as the angle of the slant cut of the rootstock. The distance where the hypocotyl is cut below the cotyledons is for example about 0.5, 1.0, 1.5, 2.0, 2.5, 3.0 or 3.5 cm.

In one embodiment the total surface areas of the two hypocotyl slant cuts is about the same size as the surface area of the rootstock slant cut. The cut area of the scions is placed against the cut area of the rootstock and held in place by fastening means until healing has occurred.

In one embodiment the slant cuts on the scions are such that each scion is in (vascular) contact with the slant cut area of the rootstock. The hypocotyl of the scions may also be cut with two or more slant cuts, to create a wedge or pointed tip on the scions, which allows vascular contact between each scion and rootstock.

Fastening means may be any known fastening means, such as a grafting clip, a spring clam, grafting tape, grafting foil, tubes or others. For cucurbits special grafting clips having been developed, which are available in different sizes, see e.g. Brinkman (Royal Brinkman International; www.brinkman.com).

The double grafted seedlings are then placed into a healing chamber for 1, 2, 3, 4, 5, 6, 7 or more days to allow healing to occur, before acclimatization and transplanting can be carried out. In one embodiment the seedlings are placed in the healing chamber for at least about 3 days.

In one aspect the method further comprises growing the double grafted seedlings into mature plants, i.e. acclimatizing the seedlings and transplanting the seedlings into the field.

### Double Stem Insertion Graft Method

Provided is a method for making a double grafted watermelon seedling, comprising the steps of:
a) Providing a rootstock comprising two cotyledons,
b) removing the meristem and shoot region of the rootstock,
c) making an incision in the stem between the two cotyledons,
d) providing a triploid watermelon scion and a diploid pollenizer watermelon scion having two slant cuts in the hypocotyl to create a pointed tip on each scion (optionally having only one slant cut in the hypocotyl)
e) inserting a triploid watermelon scion and a diploid pollenizer watermelon scion into the incision in the stem (optionally, in case the scions have only one slant cut end, in such a way that the slant sides of each of the scions face(s) towards the outside of the rootstock),
f) attaching fastening means around the two scions and the rootstock and
g) allowing healing to occur.

As mentioned previously, instead of a triploid and diploid scion, also two triploid or two diploid scions may be used in this method. The method then is a method for making a double grafted watermelon seedling, comprising the steps of:
a) Providing a rootstock comprising two cotyledons,
b) removing the meristem and shoot region of the rootstock,
c) making an incision in the stem between the two cotyledons,
d) providing two watermelon scions, each having two slant cuts in the hypocotyl to create a pointed tip on each scion (optionally having only one slant cut in the hypocotyl)
e) inserting both watermelon scions into the incision in the stem (optionally, in case the scions have only one slant cut end, in such a way that the slant sides of each of the scions face(s) towards the outside of the rootstock),
f) attaching fastening means around the two scions and the rootstock and
g) allowing healing to occur.

Step a) thus involves sowing a suitable rootstock seed and allowing the seedling to grow until two cotyledons and preferably the first true leaf are present (and optionally the second true leaf is developing). The rootstock shoots and meristem region is removed, e.g. with a razor blade or by hand) and a short cut is made vertically in the stem between the two rootstock cotyledons. The cut may be about 0.4, 0.5 or 0.6 cm deep. The cut can be made with a razor blade.

To make the scions for grafting, a triploid watermelon seed is sown and a diploid watermelon seed is sown (or two triploid watermelon seeds, or two diploid watermelon seeds, are sown) and these are allowed to grow until they have two cotyledons and preferably the first true leaf is developing or has developed, and optionally the second true leaf is starting to develop.

The hypocotyl is then cut below the cotyledons with (at least) two slant cuts (to create a pointed tip at the hypocotyl end), or optionally one slant cut (using for example a razor blade). Each scion is placed into the slit made in the rootstock stem. If only one slant cut is present at the hypocotyl end the cut side (slant side) preferably faces towards the outside of the rootstock stem. When inserting the scions, care should be taken to not create a tear in the stem tissue below the cut. The three plant parts (two scions and rootstock) are then fastened, e.g. using fastening tape or a clip or the like, until healing has occurred.

The distance where the hypocotyl is cut below the cotyledons is for example about 0.5, 1.0, 1.5, 2.0, 2.5, 3.0 or 3.5 cm.

The double grafted seedlings are then placed into a healing chamber for about 1, 2, 3, 4, 5, 6, 7 or more days to allow healing to occur, before acclimatization and transplanting can be carried out. In one embodiment the seedlings are placed in the healing chamber for at least about 3 days.

In one aspect the method further comprises growing the double grafted seedlings into mature plants, i.e. acclimatizing the seedlings and transplanting the seedlings into the field.

Double grafted seedlings and double grafted plants obtainable by any of the grafting methods described herein are encompassed as embodiments of the invention. These seedlings thus contain at least two genetically distinct plant parts (two genetically identical watermelon scions and one rootstock), or preferably at least three genetically distinct plant parts (two genetically distinct watermelon scions and one rootstock, e.g. a triploid and a diploid scion or two different diploid scions or two different triploid scions). Genetical distinction between different plant parts can be seen due to differences in morphological and/or physiological characteristics and/or through DNA analysis of the different plant parts, such as fingerprinting analysis.

### Time-Lines

Table 1 shows an example of the time-line for double grafting, using average data for rootstock (Cucurbita hybrid type) early winter season. Adaptations may need to be made depending on season, facilities available, rootstock plants used, triploid and pollenizer plants used, etc. For example, the pollenizer may need to be sown earlier than the triploid to reach the desired size for the scion. Likewise the rootstock may need to be sown at a different time point to reach the desired size for grafting.

TABLE 1

| | | |
|---|---|---|
| Day 0 | Sow triploid seed | Sowing and |
| | (germination chamber: about 28° C., about | growing |
| | 85% RH, 48-72 hours) | |
| Day 2 | Sow pollinizer seed | |
| | (germination chamber: about 26° C., about | |
| | 85% RH, 48-72 hours) | |
| Day 5 | Sow rootstock seed | |
| | (germination chamber: about 26° C., about | |
| | 85% RH, 48-72 hours) | |
| Day 14 | Double graft rootstock, triploid and pollinizer | Grafting and |
| | (healing chamber: about 24-28° C., about | Healing |
| | 95% RH - gradually decreasing) | |
| Day 19 | Post-healing acclimatization | |
| | (day time 24-28° C.; night time not below | |
| | 16° C.) | |
| Day 21 | Nursery | Nursery |
| | (night time not below 16° C.) | |
| Day 32 | Acclimatization before transplanting | |
| | About 3 days conditions close to field | |
| | conditions | |
| Day 35 | Planting in the field | Field |

### Field Arrangements

Double grafted plants are ready for transplanting when the roots are sufficiently developed to permit removal from the well of the tray with the entire soil or growing mix volume intact. This will generally require about four to six weeks from sowing or seeding, depending on well size, light and temperature conditions.

As mentioned above, the trays comprising the double grafted and triploid hybrid watermelon seedlings are preferably arranged in the same way as the plants are to be arranged in the field. See also FIG. 4-C, where one double grafted plant (DG) is in the transplant tray and in the field at every fourth position in a row of triploid watermelon plants (i.e. about 25% of plants in the field are double grafted).

In one embodiment a method for producing triploid watermelon fruits in a field is provided, said method comprising:
(a) interplanting double grafted plants according to the invention and triploid hybrid plants in one field,
(b) allowing pollination of flowers of the triploid hybrid plants (for example with pollen of the double grafted plants),
(c) harvesting triploid fruits.

Step c) may comprise harvesting fruits produced on the triploid hybrid plants and optionally fruits produced on the double grafted plants. Depending on the scions used on the double grafted plants, the fruits on the double grafted plants may be triploid, seedless fruits and/or diploid (seeded) fruits.

In step a) different planting schemes can be applied. Basically, in the traditional triploid production field, the pollenizer can be replaced by a double grafted plant according to the invention. The double grafted plants may be interplanted at regular intervals in the same row (e.g. 1, 2, 3 or 4 consecutive triploid plants followed by one double grafted plant, etc.), or rows of triploid hybrids and double grafted plants may alter at certain intervals (e.g. 1, 2, 3 or 4 rows of triploids followed by one row of double grafted plants). Alternatively, the triploids are planted in rows and the double grafted plants are planted at regular intervals in-between rows (as for example shown in FIG. 4-B, wherein the ‘Pol’ may be a double grafted plant according to the invention).

Thus, in principle the same field arrangement as for traditional triploid watermelon production can be used.

Thus, a field may comprising triploid hybrid watermelon seedlings and the double grafted seedling according to the invention in a ratio of 5:1, 4:1, 3:1, 2:1 or 1:1.

In one embodiment each of 5, 4, 3, 2 or 1 consecutive plants are triploid hybrid watermelon seedling followed by at least one double grafted seedling according to the invention. Optionally each of the consecutive triploid hybrid plants may also be followed by 2 or 3 double grafted seedling according to the invention.

In another embodiment the field comprises rows of only triploid hybrids and rows of only double grafted seedlings, whereby the ratio of triploid rows to double grafted rows is 5:1, 4:1, 3:1, 2:1, or optionally 1:1.

As mentioned, the hybrid triploids may be non-grafted or single-grafted plants. Any triploid hybrid may be used, such as known triploid hybrid varieties.

Optimal distances between plants and between rows may vary greatly depending on location, growing conditions, etc. Distances between plants may thus be any distance, such as about 3 feet (about 90 cm), about 4 feet (about 120 cm), about 5 feet (about 150 cm) or about 6 feet (about 180 cm) or more.

In step b) pollination is allowed to occur, whereby the female flowers of the triploid hybrid plants are pollinated with pollen of the (diploid scion) of the double grafted plants Likewise, the female flowers of the triploid scion of double grafted plant will be pollinated with pollen of the diploid scion of the double grafted plants. Pollination of triploid flowers results in seedless, triploid fruits, which can then be harvested in step c). Also pollination of the diploid scion will result in diploid, seeded fruits. Thus diploid fruits (which may also be marketable, depending on the diploid scion used) may be harvested from the double grafted plants. If the diploid scion is a non-marketable pollenizer the diploid, seeded fruits can be harvested and discarded and/or left in the field. For example, pollenizers comprising the explosive -rind-gene produce non-marketable fruits, which may be left on the plants and/or in the field.

Pollination is usually done by bees, and bee hives can be provided to the fields unless sufficient wild bees are naturally present. Pollination can also be performed by manual or mechanical means. Harvest at maturity may be done by hand or mechanized.

Using double grafted plants according to the invention, farmers can save more than 100 US dollars per acre field compared to traditional triploid fields, using normal pollenizers (see Examples).

The diploid, seeded fruits may be distinguished from the triploid seedless fruits based on the smaller fruit size of the diploid fruit, and/or alternatively by a different rind pattern.

Preferably harvested diploid and triploid fruit are placed into different containers. Thus, in one embodiment a container comprising solely triploid fruits or solely diploid fruits is provided. Any type of container may be used, e.g. cartons, boxes, etc.

The following non-limiting examples illustrate the invention.

### EXAMPLES

### Example 1

Three field planting schemes are referred to herein below, indicated as Scheme 1, Scheme 2 and Scheme 3 and are shown in FIG. 4 A, B and C (respectively).

Scheme 1 is an in-row planting scheme comprising 25% pollenizer plants (indicated as ‘2n’). Triploid plants are indicated as ‘3n’.

Scheme 2 is a scheme comprising 25% pollenizer plants (‘Pol’) interplanted between rows of triploids plants (indicated as ‘3n’)

Scheme 3 is an in-row planting scheme comprising 25% double grafted plants according to the invention. Triploid plants are indicated as ‘3n’. In Scheme 3 two sub-schemes are differentiated, one with a double grafted plant comprising a marketable pollenizer (producing marketable seeded fruits) and one a double grafted plant comprising a dedicated pollenizer (producing non-marketable fruits), indicated as Scheme 3A and 3B respectively.

| | | | | No. of | No. of | |
|---|---|---|---|---|---|---|
| | | | | pollenizer | single- | No. of |
| | | | Distance | or double | grafted | plants |
| | | Distance | between | grafted | triploid | per |
| | | between | plants | plants per | plants per | area |
| Scheme | Area unit | rows (feet) | (feet) | area unit | area unit | unit |
| 1 | Acre | 7 | 6 | 259 | 778 | 1037 |
| 2 | Acre | 7 | 6 | 259 | 1037 | 1296 |
| 3A | Acre | 7 | 6 | 259 | 778 | 1037 |
| 3B | Acre | 7 | 6 | 259 | 778 | 1037 |

Detailed cost per each planting scheme (in US dollar; $):

| | | | | Plant | | |
|---|---|---|---|---|---|---|
| | No. of | No. of | | costs per | | |
| | pollenizer | single- | | area unit | Plant | |
| | or double | grafted | No. of | for | costs | Total |
| | grafted | triploid | plants | pollenizers | per area | costs |
| | plants per | plants per | per | or double | unit for | per |
| Scheme | area unit | area unit | area unit | grafted | triploids | area unit |
| 1 | 259 | 778 | 1037 | $133 | $458 | $591 |
| 2 | 259 | 1037 | 1296 | $140 | $610 | $751 |
| 3A | 259 | 778 | 1037 | $174 | $458 | $632 |
| 3B | 259 | 778 | 1037 | $181 | $458 | $639 |

Comparing Scheme 3 versus Scheme 2 fields, farmers can save between 111$ and 119$ per acre, depending if the double grafted plants contain a marketable diploid watermelon pollinizer or a dedicated (non-marketable) pollenizer.

During the grafting process double graft plants will be placed in the transplant trays in the needed proportion and desired distribution, whereby planting is simplified and the risk of mistakes is reduced.

| | No. of | No. of | | | | |
|---|---|---|---|---|---|---|
| | pollenizer | single- | | Plant | | Total |
| | or double | grafted | No. of | hand | | costs of |
| | grafted | triploid | plants | labour | Planting | planting |
| | plants per | plants per | per | costs per | costs per | and hand |
| Scheme | area unit | area unit | area unit | unit ($) | area unit | labour ($) |
| 1 | 259 | 778 | 1037 | $0.08 | $83 | $674 |
| 2 | 259 | 1037 | 1296 | $0.08 | $104 | $855 |
| 3A | 259 | 778 | 1037 | $0.08 | $83 | $715 |
| 3B | 259 | 778 | 1037 | $0.08 | $83 | $722 |

Total cost analysis increases the difference between Scheme 2 and Scheme 3 further.

### Example 2

In the experiment below the double insertion grafting method (FIG. 1) was used to produce double grafted seedlings and plants. WM means watermelon and RT means rootstock.

| Day | | | | |
|---|---|---|---|---|
| count | | Day/Month | Actions | Remarks |
| 0 | Tuesday | 29-Jan | Triploid watermelon | Night temp not below |
| | | | Sowing, Pixie F1 | 16 C. |
| 1 | Wednesday | 30-Jan | | |
| 2 | Thursday | 31-Jan | | |
| 3 | Friday | 1-Feb | Diploid watermelon | Night temp not below |
| | | | sowing, Red Star F1 | 16° C. except for |
| | | | Rootstock sowing, | rootstock, kept not |
| | | | Shintosa Camelforce F1 | below 11° C. to avoid |
| | | | | fast growth |
| 4 | Saturday | 2-Feb | | |
| 5 | Sunday | 3-Feb | | |
| 6 | Monday | 4-Feb | | |
| 7 | Tuesday | 5-Feb | | |
| 8 | Wednesday | 6-Feb | | |
| 9 | Thursday | 7-Feb | | |
| 10 | Friday | 8-Feb | | |
| 11 | Saturday | 9-Feb | Grafting, place in healing | Double insertion |
| | | | chamber (healing tunnel in | method (FIG. 1) and |
| | | | this case, small tunnels of | cutting of rootstock |
| | | | 70 cm high, with heated | roots. |
| | | | pad) | WM with almost one |
| | | | | true leaf, RT with two |
| | | | | true leaves. |
| | | | | Drive the plant into a |
| | | | | pot (24 per tray) with |
| | | | | well irrigated peat. |
| | | | | Place in healing |
| | | | | chamber 24° c. |
| | | | | temperature, 95% |
| | | | | relative humidity RH. |
| 12 | Sunday | 10-Feb | | |
| 13 | Monday | 11-Feb | | |
| 14 | Tuesday | 12-Feb | | |
| 15 | Wednesday | 13-Feb | Open 2 cm plastic bands | Reduce RH to 80% |
| | | | of tunnel | |
| 16 | Thursday | 14-Feb | | |
| 17 | Friday | 15-Feb | Open 4 cm plastic bands | Keep on reducing RH |
| | | | of tunnel | gradually to balance |
| | | | | with external relative |
| | | | | humidity. |
| 18 | Saturday | 16-Feb | | |
| 19 | Sunday | 17-Feb | Remove plastic cover, | |
| | | | keep plants under shade. | |
| 20 | Monday | 18-Feb | Remove shade, remove | Once removed from |
| | | | heating pad or bring the | healing tunnel, keep in |
| | | | plants to the nursery. | greenhouse with night |
| | | | | temperature not below |
| | | | | 16° C. |
| 21 | Tuesday | 19-Feb | | |
| 22 | Wednesday | 20-Feb | | |
| 23 | Thursday | 21-Feb | | |
| 24 | Friday | 22-Feb | | |
| 25 | Saturday | 23-Feb | | |
| 26 | Sunday | 24-Feb | | |
| 27 | Monday | 25-Feb | | |
| 28 | Tuesday | 26-Feb | | |
| 29 | Wednesday | 27-Feb | | |
| 30 | Thursday | 28-Feb | Reduce night heating or | |
| | | | place in a colder place in | |
| | | | the greenhouse. | |
| 31 | Friday | 29-Feb | | |
| 32 | Saturday | 1-Mar | Ready, deliver plants to | |
| | | | the field, planting | |
| 33 | Sunday | 2-Mar | | |
| 34 | Monday | 3-Mar | | |
| 35 | Tuesday | 4-Mar | | |
| 36 | Wednesday | 5-Mar | | |
| 37 | Thursday | 6-Mar | | |

The double grafted seedlings (see FIGS. 1-D and 1-E) healed well and grew well in the nursery and in the field.

All documents (e.g., patents and published patent applications) mentioned in this specification are hereby incorporated by reference in their entirety.

1. A plant seedling comprising two scions of the species Citrullus lanatus joined to a single rootstock by grafting.

2. The plant seedling of claim 1, wherein one of the scions is from a triploid watermelon plant and the other scion is from a diploid watermelon plant.

3. The plant seedling according to claim 1, wherein both scions are from a diploid watermelon plant or both scions are from a triploid watermelon plant.

4. The plant seedling according to claim 2, wherein the diploid watermelon plant is a watermelon pollenizer plant.

5. The seedling according to claim 1, wherein the rootstock is a Cucurbita species rootstock, a bottle gourd rootstock, wax gourd rootstock, an interspecific hybrid of two Cucurbita species, a Citrullus species rootstock, or a transgenic rootstock.

6. The seedling according to claim 5, wherein the Cucurbita species rootstock is Cucurbita maxima, Cucurbita moschata, Cucurbita pepo, or an interspecific hybrid between C. maxima and C. moschata.

7. A plant grown from a seedling according to claim 1.

8. A tray comprising the seedling of claim 1.

9. The tray according to claim 8 further comprising triploid hybrid watermelon seedlings and the plant seedling comprising two scions of the species Citrullus lanatus joined to a single rootstock by grafting in a ratio of 5:1, 4:1, 3:1, 2:1 or 1:1.

10. The tray according to claim 9, wherein each of 5, 4, 3, 2 or 1 consecutive wells comprise a triploid hybrid watermelon seedling followed by one well comprising the seedling comprising two scions of the species Citrullus lanatus joined to a single rootstock by grafting.

11. A method for producing triploid watermelon fruits in a field comprising:
(a) interplanting double grafted seedlings comprising two scions of the species Citrullus lanatus joined to a single rootstock by grafting and triploid hybrid seedlings in one field,
(b) allowing pollination of flowers of the triploid hybrid plants,
(c) harvesting triploid fruits.

12. The method according to claim 11, wherein the ratio of triploid hybrid seedlings to double grafted seedlings is 5:1, 4:1, 3:1, 2:1 or 1:1.

13. A method for making a double grafted watermelon seedling, comprising the steps of:
(a) removing the rootstock meristem from a rootstock comprising two cotyledons,
(b) making a hole at the base of each cotyledon,
(c) inserting a watermelon scion into one hole and inserting a watermelon scion into the other hole, and
(d) allowing healing to occur.

14. A method for making a double grafted watermelon seedling, comprising the steps of:
(a) removing the rootstock meristem and optionally one cotyledon from a rootstock comprising two cotyledons,
(b) making a hole at the base of the cotyledon and making a hole in the upper part of the rootstock stem,
(c) inserting a watermelon scion into one hole and inserting a watermelon scion into the other hole, and
(d) allowing healing to occur.

15. The method according to claim 13, wherein the scions in step d) are i) two diploid watermelon scions, ii) two triploid watermelon scions or iii) a diploid watermelon scion and a triploid watermelon scion.

16. A method for making a double grafted watermelon seedling, comprising the steps of:
(a) removing one cotyledon and the rootstock meristem and shoot with a slant cut from a rootstock comprising two cotyledons,
(b) providing two watermelon scions having a slant cut hypocotyl end,
(c) placing the slant cut hypocotyl end of each watermelon scion onto the slant cut surface of the rootstock,
(d) attaching a fastener around the two scions and the rootstock, and
(e) allowing healing to occur.

17. The method according to claim 16, wherein the two watermelon scions are i) two diploid watermelon scions, ii) two triploid watermelon scions or iii) a diploid watermelon scion and a triploid watermelon scion.
